(19) United States
(12) Patent Application Publication
(10) Pub. No.: US 2011/0152506 A1
(43) Pub. Date: Jun. 23, 2011

(54) Method for Purifying Erythropoietin

(75) Inventors: Wolfgang Wienand, Bergisch-Gladbach (DE); Franz-Rudolf Kunz, Gelnhausen (DE); Dietmar Reichert, Eschau (DE); Wilfried Eul, Alzenau (DE); Rudolf Hanko, Dusseldorf (DE); Christian Birr, Neckargemund (DE); Monika Singhofer-Wowra, Ladenburg (DE); Dagmar Schopohl-König, Dresden (DE); Lars Faber, Ober-Ramstads (DE)

(73) Assignee: EVONIK DEGUSSA GMBH, Essen (DE)

(21) Appl. No.: 12/996,203

(22) PCT Filed: May 28, 2009
(86) PCT No.: PCT/EP09/56544

§ 371 Date: Feb. 5, 2011

(30) Foreign Application Priority Data
Jun. 4, 2008 (DE) 102008002209.8

(51) Int. Cl. C07K 14/505

(52) U.S. Cl. 530/397

(57) ABSTRACT

The present invention relates to a method for preparing erythropoietin, wherein culture supernatant of erythropoietin-producing eukaryotic cells containing erythropoietin are subjected to the following steps: a) Removing the cell components; and b) treating the product from a) to the following chromatography steps in the sequence indicated i) reversed phase chromatography; ii) anion exchange chromatography; iii) hydroxyapatite chromatography.

The present invention relates to a method for purifying erythropoietin, where erythropoietin-comprising culture supernatant of erythropoietin-producing eukaryotic cells is worked up by specific chromatographic purification steps which are carried out in succession in a specified manner.

Erythropoietin, abbreviated to EPO, is a glycoprotein which stimulates the formation of erythrocytes in the bone marrow. EPO is formed predominantly in the kidneys from where it reaches its target via the blood circulation. In the case of kidney insufficiency, the damaged kidneys produce not enough EPO or none at all, as a result of which the bone marrow stem cells give rise to too few erythrocytes. This so-called renal anemia can be treated by administering EPO in physiological amounts, which stimulate the formation of erythrocytes in the bone marrow. The EPO used for the administration can either be obtained from human urine or generated by genetic engineering methods. Since EPO only occurs in traces in the human body, the isolation of EPO from the natural source is virtually impossible for therapeutic applications. Therefore, genetic engineering methods are the only economical option of producing the substance in substantial amounts.

The recombinant production of erythropoietin has been possible since the human erythropoietin gene was identified in 1984. Since the beginning of the 1990s, various pharmaceuticals have been developed which contain human erythropoietin which has been produced by the genetic engineering route in eukaryotic cells, mainly in CHO cells (Chinese Hamster Ovary cells). The production of recombinant human erythropoietin is described for example in EP-A-0 148 605 and EP-A-205 564.

The recombinant production of erythropoietin is usually done in CHO host cells. While these host cells once used to be grown in culture media to which fetal calf serum and sometimes also bovine insulin had been added, they are currently routinely grown in a serum- and protein-free medium. In this manner, the risk of contamination with bovine proteins, bovine viruses, bovine DNA or other undesired substances of bovine origin can be avoided in its entirety. Suitable serum- and protein-free media for growing eukaryotic cells are offered by a variety of commercial manufacturers, for example the medium MAM-PF2, which is sold by, inter alia, Bioconcept, Allschwil, Switzerland, or the media DMEM and DMENU12, which are sold for example by Invitrogen/Gibco, Eggenstein, Germany.

Various chromatographic purification methods for erythropoietin have also already been described in the prior art. EP-A-0 228 452 describes a method of purifying biologically active erythropoietin from a fluid, comprising the chromatographic steps anion exchange chromatography and reversed-phase chromatography.

EP-A-0 267 678 describes the purification of an erythropoietin produced in serum-free culture, with a dialysis, an ion-exchange chromatography, a preparative reversed-phase HPLC and a gel-filtration chromatography being carried out in succession. Here, the gel-filtration chromatography step can be replaced by ion-exchange chromatography. Likewise, it is proposed to carry out a dye-affinity chromatography on a Blue trisacryl column before the (first) ion-exchange chromatography.

EP-A-0 830 376 describes a method of purifying erythropoietin in which EPO from the culture supernatant is subjected to dye-affinity chromatography in the first step of the chromatographic purification. In the second step, this is followed by a chromatography on a hydrophobicized carrier followed by a hydroxyapatite chromatography. This is followed by a reversed-phase HPLC, followed by an anion-exchange chromatography as the last chromatographic step.

EP-A-1 127 063 describes a purification method for erythropoietin which comprises the following steps: differential precipitation, hydrophobic interaction chromatography, diafiltration, anion-exchange chromatography, cation-exchange chromatography and size-exclusion chromatography. The individual purification steps are carried out in the order mentioned in EP-A-1 127 063. In one variant of the method, the purification comprises the following steps: differential precipitation, hydrophobic interaction chromatography, diafiltration, anion-exchange chromatography, cation-exchange chromatography, a further diafiltration and size-exclusion chromatography. In each case, the method provides, in the first step, a precipitation followed by centrifugation. Likewise, gel filtration is mandatorily provided to conclude the chromatographic purification.

The international application WO-A-03/045996 describes a purification method for EPO comprising an anion-exchange chromatography followed by a reversed-phase chromatography and a further anion-exchange chromatography. The second anion-exchange chromatography is followed by a size-exclusion chromatography using a gel filtration medium.

The purification of erythropoietin is also subject matter of EP-A-0 428 267. Here, a chromatography is carried out on a Q Sepharose column followed in some cases by reversed-phase chromatography and gel filtration.

WO2005/121173 provides a method for purifying EPO which has been produced by means of fermentative methods. This method is based on chromatographic purification with at least four different chromatographic separation methods. The first anion-exchange chromatography is followed by an affinity chromatography, a hydrophobic-interaction chromatography and a hydroxyapatite chromatography, the order of these 3 last-mentioned chromatography types being as desired. Finally, anion-exchange chromatography is employed again.

Thus, at least 5 chromatographic steps are required when using these methods for producing EPO which meets the purity criteria stipulated by the European Pharmacopoeia. Criteria for a suitable EPO are, inter alia, a content of heterologous proteins which are derived from the host cell of <100 ppm, a content of DNA from the host cell of <100 pg/mg EPO and, finally, a composition which meets the standard in respect of the isoform composition (Ph. Eur.; 01/2002:1316).

The object of the present invention is to describe a further purification method. The intention is to obtain an EPO end product which meets the standard defined in the European Pharmacopoeia (Ph. Eur.; 01/2002:1316) and/or the Guidance on Biosimilar Medicinal Products Containing Recombinant Erythropoietins (EMEA/CHMP/94256/2005) and which can be employed in a suitable manner in a technical, in particular fermentative, EPO production. In particular, it is intended that the present method be superior to the prior-art methods from the economical point of view. Furthermore, it is intended that the method according to the invention manage with fewer chromatographic purification steps in combination with a negligible deterioration of the purification performance and that it dispense with specific, technically complicated chromatographic steps, such as, for example, affinity chromatography.

The technical problem is solved by a method for purifying erythropoietin, where erythropoietin-comprising culture supernatant of erythropoietin-producing eukaryotic cells is subjected to the following steps:

a) removing cell components; and
b) treating the product of a) by the following chromatographic steps in the stated order:
i) reversed-phase chromatography;
ii) anion-exchange chromatography;
iii) hydroxyapatite chromatography.

In a preferred embodiment of the method, no further chromatography methods are employed before step a) and between steps a) to b iii). In addition, it is furthermore preferred that no other chromatographic methods are also employed after step b iii).

The method according to the invention provides for the propagation of an erythropoietin product which meets the standard defined in the European Pharmacopoeia (Ph. Eur.; 01/2002:1316) and/or the Guidance on Biosimilar Medicinal Products Containing Recombinant Erythropoietins (EMEA/CHMP/94256/2005). In this context, the thus prepared erythropoietin meets the following criteria: a content of heterologous proteins which are derived from the host cell of <100 ppm, a content of DNA from the host cell of <100 pg/mg EPO and, finally, a composition which meets the standard in respect of the isoform composition (Ph. Eur.; 01/2002:1316). The EPO product obtained furthermore has a biological activity of at least 150 000 IE/mg in the bioassay. Moreover, the band structure in the IEF and glycosylation pattern correspond to a commercial Erypo® product.

By the fact that, in a method of purifying the fermentation supernatant of a biotechnological EPO production from mammalian cell cultures, the fermentation supernatant which has been purified from cell components is treated by the following chromatography steps in the stated order
i) reversed-phase chromatography (RP chromatography
ii) anion-exchange chromatography (DEAE chromatography
iii) hydroxyapatite chromatography (HA chromatography)
   one arrives at the solution to the problem posed in an extremely advantageous and yet surprising manner. A person skilled in the art seeking to solve the problem as defined would, in the light of the existing prior art—in particular WO2005/121173, according to which an RP chromatography should if possible be avoided—not have considered with an expectation of success that reducing the number of required chromatography steps for purifying EPO that meets the standard is possible. The results are less equipment, personnel and materials required and also a saving of time, but above all maximum safety regarding viral contaminations. Also, the method according to the invention allows fewer adjuvants and only acceptable organic solvents such as, for example ethanol or 2-propanol, to be used. This aim is achieved in a suitable manner by carrying out the abovementioned chromatographic step in the stated order.

The chromatographic principles exploited in the method according to the invention are known from the literature and known to a person skilled in the art (Meyer, Praxis der Hochleistungs-Flüssigchromatographie [Practical high-performance liquid chromatography], Wiley-VCH Weinheim 2004; Unger, Handbuch der HPLC [HPLC Handbook], part 1 and 2, GIT Verlag Darmstadt 1994). Furthermore, more advanced and detailed information on the chromatography media can be found in the product information of the respective manufacturers or suppliers.

Furthermore, it is preferred that the eukaryotic erythropoietin-producing cells are mammalian cells, preferably human cells and especially preferably Chinese hamster ovary cells (CHO) which express human recombinant erythropoietin.

A further preferred method provides that those eluate fractions whose band pattern and glycosylation pattern do not correspond to the reference material are additionally subjected to a further anion-exchange chromatography according to ii) and/or hydroxyapatite chromatography according to iii).

Furthermore, it is preferred that an ultrafiltration is carried out after steps i), ii) and/or iii).

In a further preferred method, step a) comprises a microfiltration and a subsequent ultrafiltration.

In a preferred manner, the reversed-phase chromatography is carried out on a carrier material as the stationary phase, which is selected from the group consisting of C₁- to C₈-modified silica gels, hydrophobicized polymeric carriers based on polystyrene/divinylbenzene and hydrophobicized monolithic phase materials on silica gel or polymer basis, and an aqueous alkanol solution is used as the eluent. It is especially preferred to use, as the eluent, ethanol or 2-propanol or mixtures of these, and very especially preferably 2-propanol, with suitably buffered aqueous systems.

In a further preferred method, the anion-exchange chromatography is performed on a carrier material as the stationary phase with functional groups which are selected among diethylaminoethyl groups (DEAE), quaternary aminoethyl groups (QAE), quaternary ammonium groups or dimethylaminoethyl groups (DMAE).

Furthermore, it is preferred that the anion exchange chromatography comprises at least one wash step with an aqueous buffer system, preferably a buffer system based on an organic acid, in particular with an acetate buffer.

The anion-exchange chromatography especially preferably comprises at least one wash step with an aqueous buffer solution with a pH of from 3.5 to 5.5, especially preferably with a pH of from 4.0 to 5.0, and most preferably with a pH of approximately 4.5. A suitable buffer is above all an acetate buffer, preferably a sodium acetate buffer.

In a preferred manner, the eluent used in the anion-exchange chromatography is an inorganic acid in an aqueous buffer solution. In a furthermore and especially preferred manner, the eluent used in the anion-exchange chromatography is chloride ions in an aqueous buffer solution.

In a further preferred process, the hydroxyapatite chromatography comprises at least one wash step with a buffer system based on an organic acid, preferably with an acetate buffer.

It is likewise preferred to use, as the eluent in the hydroxyapatite chromatography, a buffer system which is based on an inorganic acid, especially preferably a phosphate buffer.

### ILLUSTRATION OF THE ESPECIALLY PREFERRED EMBODIMENTS

The present invention provides a process for purifying erythropoietin, where erythropoietin-comprising culture supernatant of erythropoietin-producing eukaryotic cells is subjected to the following steps:

a) removing cell components; and
b) treating the product of a) by the following chromatography steps in the stated order:
i) reversed-phase chromatography;
ii) anion-exchange chromatography;
iii) hydroxyapatite chromatography.

The reversed-phase chromatography is employed as a “capture step”. In this first purification step, EPO is concentrated from the fermentation supernatant. What is of importance here are different hydrophobic interactions between the sample molecules and the stationary phase. The less the sample components are soluble in water, i.e. the more nonpolar they are, the more they are retained. The reversed-phase chromatography can be carried out with traditional, commercially available phase materials, both on a silica base and on a polymer base, which phase materials are specifically suitable for protein analysis. Materials which are typically employed are large-pore a silica gel materials, for example 300 Å or 500 Å, with a short-chain carbon load, for example C1, C2, C3 and C4, nonporous hydrophobicized polystyrene/divinylbenzene-based polymeric carriers and, specifically, hydrophobicized monolithic phase materials, likewise on a silica gel or polymer base. The manufacturers or suppliers of these stationary phases include, inter alia, the companies Merck, Waters, GE Healthcare, Tosoh Bioscience, Bio-Rad, Dionex, YMC, Phenome-nex, Machery-Nagel and BIO Separations.

Preferred reversed-phase materials are nonporous hydrophobicized polystyrene/divinylbenzene-based polymeric carriers. Especially preferred are SOURCE 30RPC or the corresponding 15-μm-material (SOURCE 15RPC) from GE Healthcare.

Eluents which can be employed are, preferably, alcohols such as ethanol or 2-propanol or mixtures of these with suitably buffered aqueous systems. The use of 2-propanol is especially preferred since the organic solvent fraction in the eluent is markedly lower in comparison with ethanol, which means advantages in terms of safety and economy. Furthermore, 2-propanol is outstandingly suitable as solubilizer due to its miscibility and solubility properties (Unger, Handbuch der HPLC [HPLC Handbook], part 1, GIT Verlag Darmstadt 1994; Nowotny et al., Chromatographia 1988, 25, 409-412).

The anion-exchange chromatography is based on the competitive interaction of charged ions of the sample solution with the buffer medium employed. It can be carried out with conventional, commercially available anion-exchange resins with diethylaminoethyl (DEAE), quaternary aminoethyl (QAE), quaternary ammonium or dimethylaminoethyl (DMAE) functionalization. These phase materials can be obtained for example from GE Healthcare, Tosoh Bioscience, Bio-Rad or Merck. It is preferred to employ diethylaminoethyl (DEAE)-functionalized anion-exchanger resins. It is especially preferred to use TSKgel DEAE-5PW (30 μm), available from Tosoh Bioscience, in the anion-exchange chromatography. This chromatographic step is important in the light of the glycosylation pattern required in the final EPO product.

In the preferred embodiment, the anion-exchange chromatography comprises an acidic wash step (“acidic wash”), with which the basic isoforms of erythropoietin are eluted, and therefore separated, as the result of the lowering of the pH (EP-1428878). “Acidic wash” means in this context that the pH of the wash buffer is between 3.5 and 5.5, especially preferably between 4.0 and 5.0 and most preferably approximately 4.5. A suitable buffer is, above all, a sodium acetate buffer.

To carry out the hydroxyapatite chromatography, it is possible to employ traditional hydroxyapatite (also referred to as hydroxylapatite) materials. Hydroxyapatite is hexagonally crystalline calcium phosphate and is particularly suitable for the separation of proteins and other biopolymers. This purification step is carried out for removing the “phosphated” EPO molecules. It is preferred to employ CHT ceramic hydroxyapatite (Bio-Rad), especially preferably CHT ceramic hydroxyapatite type 1 (Bio-Rad).

To increase the total product yield, not only the reversed-phase chromatography, but also the anion-exchange chromatography and the hydroxyapatite chromatography may be repeated, in the sense of a reprocessing, for those eluate fractions whose product quality, in particular whose band or glycosylation pattern, does not correspond to the reference material. These are typically the leading and trailing edge fractions of the main fractions, which per se, already feature the desired product characteristics after the first purification step. The classification of the fraction quality at the different purification levels was performed using standard methods (isoelectric focusing (IEF) and high-performance anion exchange chromatography with pulsed amperometric detection (HPAEC-PAD): Lasne, Nature 2000, 405 605-635; Holländer et al., LaborPraxis 2004, 56-59; Hokke et al., Eur. J. Biochem. 1995, 228, 981-1008; Dionex Technical Note 42, 1997. The reference material used in each case is a commercially available Erypo® product (JANSSEN-CILAG).

Prior to the purification of the culture supernatant with the abovementioned chromatographic methods, a microfiltration through 1.2-μm- and 0.65-μm-filters is carried out in order to remove cells. This is followed by an ultrafiltration (cut-off 10 000 Da) of the cell-free filtrate to 1/10 of the original volume. After the ultrafiltration, the concentrated cell supernatant is filter-sterilized and employed in the first chromatography step (reversed-phase chromatography). The filtration steps, in particular filter sterilization, are known from the literature and known to a person skilled in the art (Munir, Handbuch Ultrafiltration, Behr Hamburg 1990; Ullmann Vol. 2, 10-2, 10-21 and Vol. 3 11-6; Gasper, Handbuch der industriellen Fest-Flüssig-Filtration, Hüthig Heidelberg 1990; Ullmann A16, 187-258).

It has been found that the EPO obtained by the method according to the invention meets the quality criteria as defined in the European Pharmacopoeia. In particular, the isoform composition and the glycosylation pattern correspond to the standard defined in the European Pharmacopoeia (Ph. Eur.; 01/2002:1316) or in Guidance on Biosimilar Medicinal Products Containing Recombinant Erythropoietins (EMEA/CHMP/94256/2005). The reference material is a commercially available Erypo® product (JANSSEN-CILAG).

The activity of the protein should amount to at least 100 000 IU/mg, preferably at least 125 000 IU/mg and especially preferably at least 150 000 IU/mg (see also European Pharmacopoeia 01/2002:1316).

The EPO which has been purified in accordance with the invention is preferably recombinant human erythropoietin, produced in eukaryotic cells. It is preferred to produce the recombinant EPO in mammalian cells, especially preferably in CHO cells, such as, for example, as described in EP-A-0 205 564 and EP-A-0 148 605. The fermentation is preformed in accordance with conventional protocols in commercially available culture media.

For the purposes of the present invention, erythropoietin (EPO) is understood as meaning any protein which is capable of stimulating the erythrocyte formation in the bone marrow and which can be identified unambiguously as erythropoietin in the assay described in the European Pharmacopoeia (Ph. Eur.; 01/2002:1316) (determination of the activity in polycythemic or normocythemic mice). The erythropoietin may take the form of the wild-type human erythropoietin or a variant thereof with one or more amino acid substitutions, deletions or additions. If it takes the form of a variant of erythropoietin, then it is preferred that this variant differs only in 1 to 20, preferably in only 1 to 15, especially preferably in only 1 to 10 amino acid positions from human wild-type erythropoietin as the result of amino acid substitutions, deletions or additions.

In the present context, the purification of erythropoietin or concentration of erythropoietin is taken to mean that the protein erythropoietin is obtained from a mixture in very pure form, in other words the erythropoietin present in the mixture is concentrated until essentially no other proteins are any longer present other than standard erythropoietin.

### DESCRIPTION OF THE FIGURES

FIG. 1 shows an example of the chromatographic separation of the erythropoietin peak in the reversed-phase chromatography. What is plotted is the peak intensity in the form of milli-absorption units (mAU) against the elution time (in min.) at a UV wavelength of 280 nm.

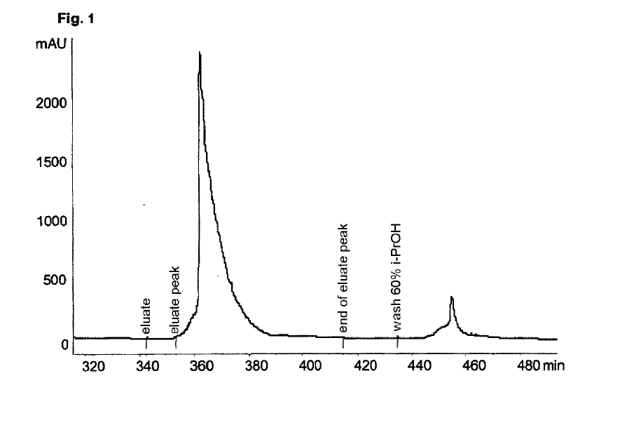

FIG. 2 shows an example of the chromatographic separation of the erythropoietin peak under the conditions of anion-exchange chromatography. What is plotted is the peak intensity in the form of milli-absorption units (mAU) against the elution time (in min.) at a UV wavelength of 280 nm.

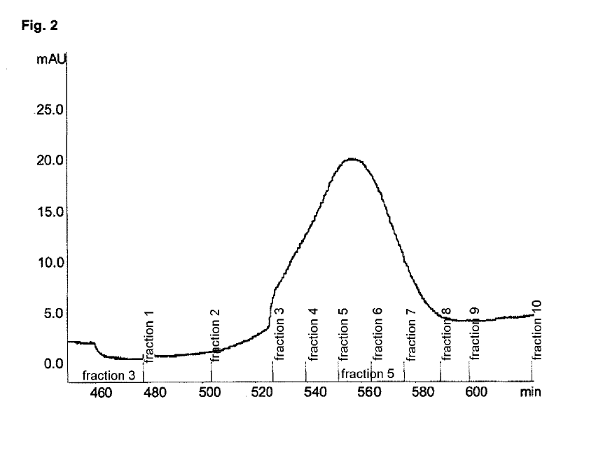

FIG. 3 shows an example of the IEF gel of the isolated EPO eluate fraction after anion-exchange chromatography in comparison with the Erypo® product.

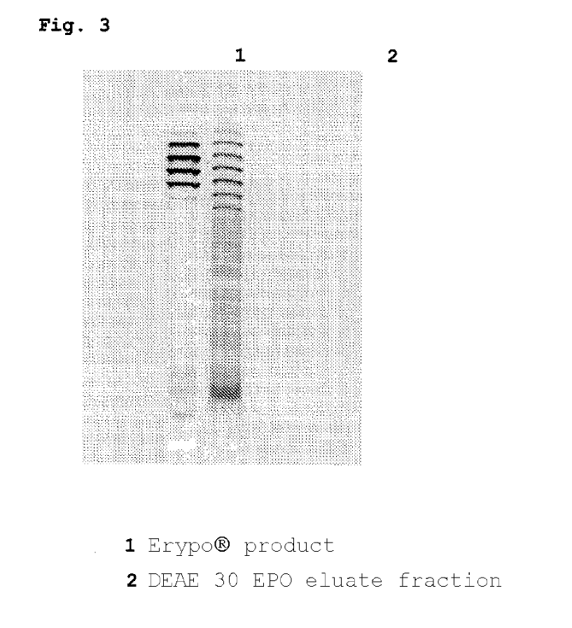

FIG. 4 shows an example of the chromatographic separation of the erythropoietin peak in the hydroxyapatite chromatography. What is plotted is the peak intensity in the form of milli-absorption units (mAU) against the elution time (in min.) at a UV wavelength of 280 nm.

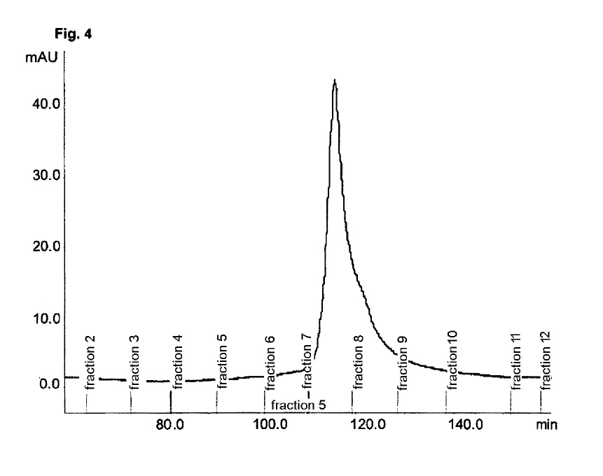

FIG. 5 shows an example of the IEF gel of the isolated EPO eluate fraction after hydroxyapatite chromatography in comparison with the Erypo® product.

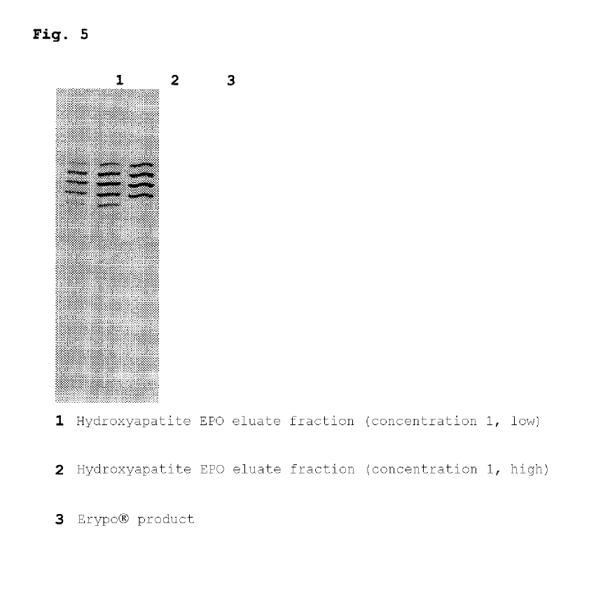

FIG. 6 shows the native glycosylation status of the purified final EPO product.

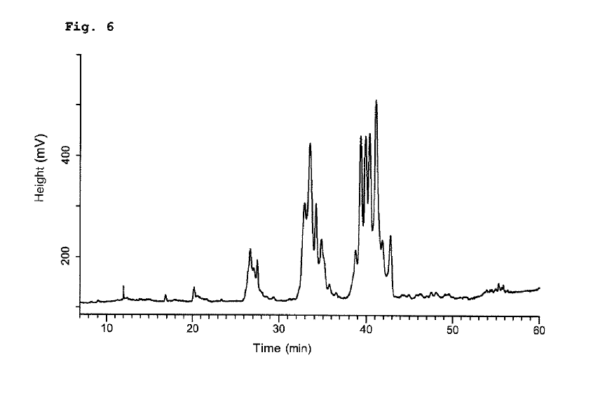

FIG. 7 shows the glycosylation state of a commercially available Erypo® product (JANSSEN-CILAG).

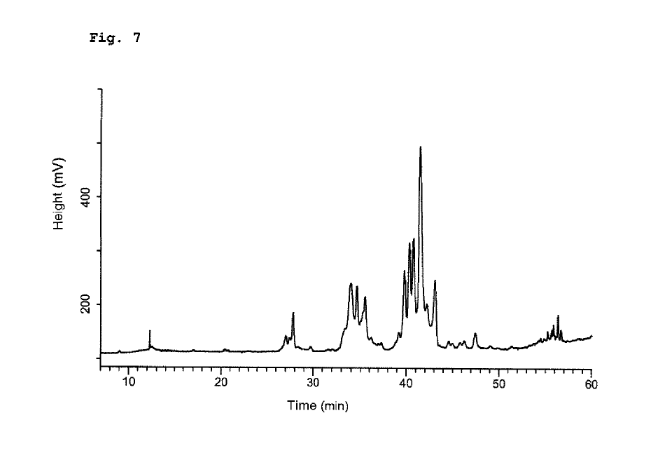

The examples which follow are intended to illustrate the invention, without imposing any limitation.

### EXAMPLES

### Example 1

### Production of EPO in CHO Cells

EPO is produced fermentatively in CHO cells. The fermentation is carried out by standard methods as they are described in the patent and scientific literature for eukaryotic cells, in particular CHO cells. Culturing takes place in a perfusion reactor in culture medium which is free from animal components. The cells are harvested continuously over a period of up to 50 days. To remove the cells, a microfiltration through a suitable filter (for example Opticap XLT30 capsule with Milligard medium 0.5-1.2 μm, Millipore and Sartobran P Midi-caps 0.45-0.65 μm, Sartorius) is carried out at a flow rate of 1-2 l/min. This is followed first by an ultrafiltration (cut-off 10 000 Da) of the cell-free filtrate (for example Ultran Pilot, polyether sulfone 4×0.45 m², Schleicher & Schull) and then by a sterile filtration (for example through Opticap filter units with 0.5 μm Milligard prefilter and 0.2 μm Durapore sterile filter, Millipore).

### Example 2

### Reversed-Phase Chromatography (“Capture Step”)

A reversed-phase chromatography on SOURCE 30RPC is carried out as the “capture step”. In this first purification step, EPO is concentrated from the fermentation supernatant. Here, several wash steps are performed. The first wash step is performed with PBS buffer (phosphate-buffered saline). The next wash step is performed with a mixture of water/isopropanol/TFA in a volume ratio of 10/2/0.1 to 10/1/0.1 at a flow rate of 40-50 ml/min. The product elution is performed with a mixture of water/isopropanol/TFA in a volume ratio of 10/4/0.1 at a flow rate of 30-40 ml/min. The flow rates were optimized and adapted in a suitable manner for this separation. Thereafter, a wash step with isopropanol/0.1% TFA solution in the volume ratio 60/40 is performed.

Directly after elution, the trifluoroacetic-acid product fraction is treated in the ratio 1:1 with a phosphate buffer (pH 10) and diluted to pH 7.2 with 15 mM sodium phosphate buffer. Now, the EPO-comprising solution, which is neutralized in that step, is filter-sterilized.

The in-process control (IPC) for the EPO concentration is carried out on an analytical RP-HPLC separation column in the TFA/acetonitrile system. The EPO yield after this chromatography step amounts to at least 50%, preferably at least 65% and especially preferably at least 80%.

### Example 3

### Anion-Exchange Chromatography

The neutralized EPO product fraction from the reversed-phase chromatography is applied at a flow rate of 20 ml/min. Now, the EPO-comprising solution is purified by three wash steps at a constant flow rate of 40 ml/min, the first and third wash step being carried out in each case at a pH of 7.2 and with 20 mM sodium acetate solution and the second at a pH of 4.5 and with a suitable sodium acetate solution. Product elution is performed under gradient conditions using a sodium acetate/sodium chloride buffer (pH 7.2), (flow rate 30-40 ml/min). In this process, the amount of buffer is increased slowly and linearly from 0 to 80%.

With the aid of isoelectric focusing (IEF), the eluate fractions are analyzed and divided into the product pool (main fraction) and edge fractions, according to the position of the bands in the IEF. The reference material used is a commercially available Erypo® product. The EPO content of the main fraction is determined by means of RP chromatography. The selected eluate fractions are concentrated by means of ultrafiltration and subjected to a buffer exchange for the subsequent hydroxyapatite chromatography. After this chromatography step, the erythropoietin yield amounts to at least 20%, preferably to at least 30% and especially preferably to at least 40%.

The isoelectric focusing is carried out on an ultra-thin-layer polyacrylamide gel. To this end, the sample solutions must be desalted and concentrated with the aid of a microcentrifugation kit (cut-off 10 000 Da) before being applied. Focusing is done at voltages of from 300-2000 V. After 5000 Vh, the development is complete. The gel is then stained with silver nitrate or Coomassie and evaluated.

### Example 4

### Hydroxyapatite Chromatography

This purification step is suitable for removing the “phosphated” EPO molecules. The main fraction of the anion-exchange chromatography is subjected to a buffer exchange into the starting buffer of the hydroxyapatite column, using ultrafiltration, and thereafter filter-sterilized.

The samples are injected onto a CHT Ceramic hydroxyapatite type 1 phase at a flow rate of 30 ml/min. The wash step is carried out with a sodium acetate buffer (pH 6.8) and sample elution with a phosphate buffer at pH 6.8 and a flow rate of 50 ml/min under gradient condition. In doing so, the amount of phosphate buffer is increased slowly and linearly from 0 to 25%.

The EPO fractions which meet the specification are present in a sodium acetate/sodium phosphate buffer and are subjected to a buffer exchange with PBS (final packaging buffer) by means of ultrafiltration and frozen at −20° C. The EPO content of the individual fractions is determined by RP chromatography. The erythropoietin yield after this chromatography step amounts to at least 30%, preferably to at least 40% and especially preferably to at least 50%.

The analysis of the glycosylation pattern by means of HPAEC-PAD is performed after the enzymatic cleavage of the carbohydrate chains from the protein, using PNGase F (Roche Diagnostics GmbH). Following isolation and desalting, the sugar residues are analyzed in a high-performance anion exchanger with pulsed amperometric detection.

The final EPO product obtained has a biological activity of at least 150 000 IU/mg in the bioassay and meets all requirements of the European Pharmacopoeia (Ph. Eur.; 01/2002:1316) or the Guidance on Biosimilar Medicinal Products Containing Recombinant Erythropoeitins (EMEA/CHMP/94256/2005). In addition, the band structure in the IEF and the glycosylation pattern correspond to that of a commercially available Erypo® product.

1-17. (canceled)

18. A method for purifying erythropoietin from a culture supernatant of erythropoietin-producing eukaryotic cells, comprising:
a) removing cell components from said culture supernatant to produce a cell free supernatant;
b) purifying said erythropoietin from the cell free supernatant of step a) by performing the following procedures in the order indicated:
i) reversed-phase chromatography;
ii) anion-exchange chromatography;
iii) hydroxyapatite chromatography.

19. The method of claim 18, wherein no further chromatography methods are employed before step a) or between steps a) to b iii).

20. The method of claim 18, wherein the erythropoietin-producing eukaryotic cells are mammalian cells.

21. The method of claim 18, wherein eluate fractions whose product quality does not correspond to a reference material are additionally subjected to a further reversed-phase chromatography step and/or anion-exchange chromatography step and/or hydroxyapatite chromatography step.

22. The method of claim 18, further comprising an ultrafiltration step carried out after steps b i), b ii) and/or b iii).

23. The method of claim 18, wherein the removal of cell components in step a) by a procedure comprising microfiltration followed by ultrafiltration.

24. The method of claim 18, wherein said reversed-phase chromatography is carried out on a carrier material as the stationary phase, said carrier material being selected from the group consisting of: C₁- to C₈-modified silica gels; hydrophobicized polymeric carriers based on polystyrene/divinylbenzene; and hydrophobicized monolithic phase materials on silica gel or polymer basis; and wherein an aqueous alkanol solution is used as the eluent.

25. The method of claim 18, wherein said anion-exchange chromatography is performed on a carrier material as the stationary phase with functional groups selected from the group consisting of: diethylaminoethyl groups (DEAE); quaternary aminoethyl groups (QAE); quaternary ammonium groups; and dimethylaminoethyl groups (DMAE).

26. The method of claim 25, wherein said anion-exchange chromatography comprises at least one wash step with an aqueous buffer system.

27. The method of claim 25, wherein said anion-exchange chromatography comprises at least one wash step with a buffer system based on an organic acid.

28. The method of claim 25, wherein said anion-exchange chromatography comprises at least one wash step with an aqueous buffer solution with a pH of from 3.5 to 5.5.

29. The method of claim 25, wherein the eluent used in said anion-exchange chromatography is an inorganic acid in an aqueous buffer solution.

30. The method of claim 25, wherein the eluent used in said anion-exchange chromatography is chloride ions in an aqueous buffer solution.

31. The method of any of claim 18, wherein said hydroxyapatite chromatography comprises at least one wash step with a buffer system based on an organic acid.

32. The method of claim 18, wherein said hydroxyapatite chromatography comprises at least one wash step with an acetate buffer.

33. The method of claim 18, wherein the eluent used in said hydroxyapatite chromatography is a buffer system based on an inorganic acid.

34. The method of claim 18, wherein the eluent used in said hydroxyapatite chromatography is a phosphate buffer.

35. The method of claim 18, wherein:
a) said reversed-phase chromatography is carried out on a carrier material as the stationary phase, said carrier material being selected from the group consisting of: C₁- to C₈-modified silica gels, hydrophobicized polymeric carriers based on polystyrene/divinylbenzene; and hydrophobicized monolithic phase materials on silica gel or polymer basis; and wherein an aqueous alkanol solution is used as the eluent;
b) said anion-exchange chromatography is performed on a carrier material as the stationary phase with functional groups selected from the group consisting of: diethylaminoethyl groups (DEAE); quaternary aminoethyl groups (QAE); quaternary ammonium groups; and dimethylaminoethyl groups (DMAE).

36. The method of claim 35, wherein no further chromatography methods are employed before step a) and between steps a) to b iii) and wherein the eukaryotic erythropoietin-producing cells are mammalian cells which express human recombinant erythropoietin.

37. The method of claim 35, further comprising an ultrafiltration step carried out after steps b i), b ii) and/or b iii).
